(19) United States
(12) Patent Application Publication
(10) Pub. No.: US 2002/0119917 A1
(43) Pub. Date: Aug. 29, 2002

(54) ANTI-INFLAMMATORY PEPTIDES DERIVED FROM C-REACTIVE PROTEIN

(76) Inventors: MATITYAHU FRIDKIN, REHOVOT (IL); ERAN J. YAVIN, REHOVOT (IL)

Correspondence Address: BROWDY & NEIMARK, 624 NINTH STREET, N.W., SUITE 300, WASHINGTON, DC, 20001, US

(21) Appl. No.: 09/117,380

(22) PCT Filed: Jan. 27, 1997
(86) PCT No.: PCT/IL97/00032

(30) Foreign Application Priority Data
Jan. 31, 1996 (IL) 116976

(51) Int. Cl. A61K 38/00

(52) U.S. Cl. 514/12; 435/183; 435/218; 435/219; 530/350

(57) ABSTRACT

A peptide corresponding to positions 89-96 of the human C-reactive protein (CRP) of the formula: Val₈₉-Thr-Val-Ala-Pro-Val-His-Ile₉₆ and modifications thereof obtained by substitution, elongation, amidation of the C-terminal or acylation of the N-terminal, inhibit in vitro the enzymatic activity of human leukocyte elastase (hLE) and/or of human leukocyte cathepsin G(hCG) and can be used for the treatment of chronic inflammation conditions such as rheumatoid arthritis, pulmonary emphysema and cystic fibrosis.

### FIELD OF THE INVENTION

The present invention relates to synthetic peptides derived from the primary sequence of the acute phase reactant C-reactive protein (CRP), which peptides inhibit in vitro the enzymatic activities of human leukocyte elastase (hLE) and human leukocyte cathepsin G (hCG), two potent serine proteases associated with tissue damage occurring in the course of several chronic inflammatory conditions. The invention further relates to anti-inflammatory pharmaceutical compositions comprising said CRP-derived peptides.

### ABBREVIATIONS

The following abbreviations will be used throughout the specification: CRP, C-reactive protein; hLE, human leukocyte elastase; hCG, human leukocyte cathepsin G; α₁-PI, α₁-protease inhibitor; ACT, α-antichymotrypsin; MeOSuc-AAPV-NA, methoxysuccinyl-Ala-Ala-Pro-Val-nitroanilide; Suc-AAPF-NA, succinyl-Ala-Ala-Pro-Phe-nitroanilide.

### BACKGROUND OF THE INVENTION

C-reactive protein (CRP) is a plasma protein classified as a major acute phase reactant due to its dramatic accumulation in the blood stream during the inflammatory response. Within a relatively short period (24-48 hr) following tissue injury or certain traumatic events, the CRP blood concentration may rise 1000-fold over the normal level to as high as 1 mg/mL (Ballue and Kushner, 1992).

CRP consists of five identical sub-units that contain each 206 amino acids bridged by a single disulfide bond and that aggregate non-covalently into a cyclic pentamer termed pentraxin. The precise biochemical function of CRP as a whole entity is still obscure. CRP was shown to bind to specific receptors on human neutrophils (K_{d˜}5×10⁻⁸ M), monocytes (K_{d˜}10⁻⁷ M), and other inflanmnatory-related cells in vitro (Ballue and Kushner, 1992).

In the laboratories of the present inventors and their collaborators it was found that following binding to neutrophils, CRP is subsequently degraded by a membrane-associated neutral serine protease, which has been characterized (Shephard et al., 1992), and by lysosomal-derived enzymes, to yield various low molecular weight peptides. Several of these peptides were identified, synthesized, and shown to be potent anti-inflammatory agents inhibiting neutrophil phagocytosis, degranulation, and superoxide ion (O₂⁻) generation (Shephard et al., 1990; Yavin et al., 1995). Superoxide ion is the parent compound of several destructive mediators that are believed to play a central role in inflarnmation-associated tissue injury (Ballue and Kushner, 1992).

The most prominent of the peptides disclosed by Shephard et al., 1990, and Yavin et al., 1995, were derived from within the primary sequence of CRP as follows: Asp₇₀-lie-Gly-Tyr-Ser₇₄, Lys₂₀₁-Pro-Gln-Leu-Trp-Pro₂₀₆, Leu₈₃-Phe-Glu-Val-Pro-Glu-Val-Thr₉₀, Val₇₇-Gly-Gly-Ser-Glu-Ile₈₂ (Shephard et al., 1990) and Asn₁₆₀-Met-Trp-Asp-Phe-Val₁₆₅, Gln₂₀₃-Leu-Trp-Pro₂₀₆, Ser₁₈-Tyr-Val-Ser-Leu-Lys₂₃ (Yavin et al., 1995). These peptides were shown by the authors to inhibit neutrophilic function, indicating that they may be capable of regulating superoxide ion production by neutrophils in vivo during the acute phase response as part of a complex protective mechanism. However, as shown in the examples of the present application, several of these peptides and additional peptides with close proximity within the primary sequence of CRP have no hLE inhibitory capability.

Human leukocyte elastase (hLE) and human leukocyte cathepsin G (hCG) are potent neutral serine proteases found in the azurophilic granules of neutrophils, which are involved in the intracellular digestion of proteins and play an important role in phagocytosis and host defense against invading organisms. In the extracellular environment, hLE is capable of degrading various connective tissue proteins including highly cross-linked elastin whereas hCG is very effective in degrading proteoglycans and collagens and has been shown to augment the elastolytic capability of hLE (Groutas, 1987).

The release of enzymes into the extracellular medium by activated neutrophils is normally controlled by several potent inhibitors. The most specific natural inhibitors, (α₁-protease inhibitor (α₁-PI) and a-antichymotrypsin (ACT), are directed against hLE and hCG, respectively (Groutas, 1987). Imbalances in the levels of tissue proteases such as hLE and hCG, and their inhibitors, allow excess hLE and hCG to attack connective tissue, and are implicated in the severe and permanent tissue damage associated with pulmonary emphysema (Groutas, 1987), rheumatoid arthritis (Gallin et al., 1988), cystic fibrosis (Jackson et al., 1984) and several other inflammatory conditions. Major research efforts have been dedicated to develop potent inhibitors of hLE and hCG based on a wide variety of low molecular weight organic compounds (Edwards and Bernstein, 1994) such as 3,3-dialkylazetidin-2-ones, proposed as orally active β-lactam inhibitors of hLE (Finke et al., 1995).

CRP as a whole protein was reported to have no inhibitory effect on hLE (Vachino et al., 1988). In contrast, novel biologically active CRP-derived peptides, previously concealed within the inner hydrophobic region of each subunit, have been found, in accordance with the present invention, to significantly inhibit the enzymatic activities towards destructive enzymes.

### SUMMARY OF THE INVENTION

The present invention relates to synthetic CRP-derived peptides, which inhibit in vitro the enzymatic activity of hLE and hCG.

In particular, the present invention relates to a synthetic peptide capable of inhibiting in vitro the enzymatic activity of human leukocyte elastase (hLE) and/or of human cathepsin G ITCG), said peptide being selected from:

(i) a core peptide corresponding to positions 89-96 of the sequence of human C-reactive protein (CRP) of the formula:

Val₈₉-Thr-Val-Ala-Pro-Val-His-Ile₉₆

or a modification thereof characterized by:

(ii) substitution of Ile₉₆ by a hydrophobic amino acid residue;

(iii) substitution of His₉₅ by D-His or by a residue selected from Asp, Glu, Ser, Thr, Phe and Tyr, N-alkyl derivatives thereof and D-forms of the foregoing;

(iv) substitution of Val₉₄ by D-Val or by a residue selected from Ala, His and Phe, and D-forms of the foregoing;

(v) substitution of Ala₉₂ by a hydrophobic amino acid residue;

(Vi) substitution of Val₉₁ by Ala or Gly;

(vii) substitution of Thr₉₀ by a residue selected from Asn, Asp, Gln, Glu, Ala, Val and Pro;

(viii) substitution of Val₈₉ by a hydrophobic amino acid residue;

(ix) a peptide obtained by elongation of a peptide (i) to (viii) at the N- and/or C-terminal;

(x) an amide of the C-terminal of a peptide (i) to (ix); and

(ix) an N-acyl derivative of a peptide (i) to (x).

The invention further relates to anti-inflammatory pharmaceutical compositions comprising a CRP-derived peptide of the invention and a pharmaceutically acceptable carrier.

In another aspect, the invention relates to a method of treatment of an inflammatory disorder, e.g. rheumatoid arthritis, pulmonary emphysema, cystic fibrosis and other chronic inflammatory condition, which comprises administering to a patient in need thereof an effective amount of a CRP-derived peptide according to the invention.

### BRIEF DESCRIPTION OF THE DRAWINGS

FIG. 1 depicts the sequence of the human C-reactive protein (CRP).

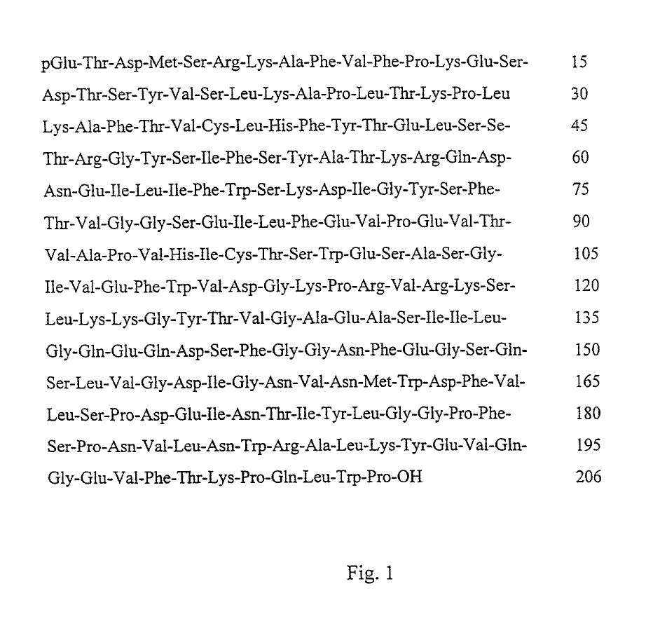

FIG. 2 is a graph of the RP-HPLC chromatograms of the degradation profile of the CRP-derived core peptide 1 of the sequence Val₈₉-Thr-Val-Ala-Pro-Val-His-Ile₉₆ by hLE at several time intervals.

### DETAILED DESCRIPTION OF THE INVENTION

The present invention provides a series of synthetic peptides derived from the sequence of CRP and to pharmaceutical compositions comprising the peptides which are anti-inflammatory by inhibiting either hLE or hCG activity, or both. These biologically active peptides can be used to inhibit hLE and/or hCG and thereby have utility in controlling tissue damage associated with chronic inflammation.

A careful examination of the sequence of CRP reveals a specific region within the protein's sequence which is similar, though not identical, to the active site of α₁-PI, the natural inhibitor of hLE, as shown below:

CRP: Ser-Phe-Thr-Val-Gly-Gly-Ser-Glu-ILe-Leu-Phe-Glu-Val-Pro-Glu-

α₁-PI Thr-Ile-Asn-Glu-Lys-Gly-Thr-Glu-Ala-Ala-Gly-Ala-Met-Phe-

CRP: Val-Thr-Val-Ala-Pro-Val₉₄ [symbol] His-Ile-Cys-s-s-Cys-Leu-His-Phe

α₁-PI Leu-Glu-Ala-Ile-Pro-Met₃₅₈ [symbol] Thr-Ile-Pro-Pro-Glu-Val-Lys-Phe

The long range sequence match between CRP and α₁-PI is shown. Bold letters denote similar amino acids, with respect to approximate steric volume, hydrophobicity and charge, or identical amino acids. From the carboxy terminal of peptide 1 (underlined) the sequence similarity is apparent although shifted from a certain position by one amino acid (e.g. Gly₇₉-Ser-Glu-Le-Leu₈₃ in CRP vs. Gly₃₄₄-Thr-Glu-Ala-Ala₃₄₈ in α₁-PI). From the amino terminal, sequence similarity may be observed through the single disulfide bridge (represented by -s-s-) found in CRP. The cleavage site (symbol [symbol] ) in α₁-PI is the Met₃₅₈-Thr₃₅₉ bond and in CRP-based peptide inhibitors is theoretically at the Val₉₄-His₉₅ bond.

The core peptide 1, Val₈₉-Thr-Val-Ala-Pro-Val-His-Ile₉₆, was chosen due to its similarity with the active site of the natural inhibitor of elastase: α₁-PI. This sequence contains the highest ratio of similar vs. dissimilar amino acids. Based on the X-ray crystallographic data obtained for hLE complexed with Turkey ovimcoid inhibitor (Bode et al., 1989), the preferred amino acid required by each subsite of hLE was formulated with respect to CRP derived analogs. For example, the enzyme's main hydrophobic pocket, in which CRP's Val₉₄ is accommodated, is large enough to contain large hydrophobic amino acids such as Ile or Leu, yet it is not large enough to contain Phe which hCG actually prefers. This core peptide 1 is cleaved specifically at the Val-His bond, making it an ideal candidate for further subsite modifications and L to D amino acid replacements.

Based on the results obtained for the core peptide 1, additional peptides 2-23 were synthesized (Table 1).

According to the invention, the residues Val₈₉, Ala₉₂ and Ile₉₆ of the core peptide 1 may be replaced by a residue of a natural aliphatic or aromatic hydrophobic amino acid, such as Leu, Ile, Val, Phe or Tyr, or of a non-natural hydrophobic amino acid, such as norleucine (Nle) and norvaline (Nva).

The residue His₉₅ may be replaced preferably by an aromatic amino acid, such as Phe or Tyr, or by Asp, Glu, Ser or Thr. D-amino acid modifications, e.g. (D)His, and N-alkylation of the peptide bond, are most beneficial in this position to prevent peptide cleavage by the enzyme.

The residue Val₉₄ is the preferred residue at this position directed towards hLE inhibition, while specificity towards hCG is gained by aromatic amino acid substitution such as Phe or His. D-amino acid modifications, e.g. (D)Val, (D)Ala, (D)Phe and (D)His are most beneficial in this position to prevent peptide cleavage by the enzyme.

Proline is important in creating a bond to the stretched, open chain peptide which increases its specific fit into the binding site of hLE and hCG (Bode et al., 1989). If the residue Pro₉₃ is replaced in this position, binding may occur in different orientations of the peptide which dramatically reduces its inhibitory activity. Thus attempt to replace it by sarcosine (i.e. N-methyl-glycine that bears some chemical resemblance to Pro, see peptide 30 in Table 2) led to a dramatic loss in inhibitory activity as compared to the core peptide 1. It is therefore not advisable to modulate this position.

The residue Thr₉₀ may be replaced by Asn, Asp, Gln or Glu, Pro, or by a medium sized hydrophobic amino acid, such as Ala or Val.

Elongation of the peptide chain of the core peptide 1 or of a modified core peptide obtained by substitution of one or more amino acid residues as described above, leads to augmentation of inhibitory activity both towards hLE (see peptides 2, 3, 3a, 3b, 3c, 4 and 5) and towards hCG (see peptides 2, 3, 3a, 3b and 3c). The C-terminus addition of Cys₉₇-Thr₉₈ to peptide 3 increases inhibitory activity towards both enzymes (peptide 3a) while the additional C-terminus fragment, Ser₉₉-Trp-Glu-Ser-Ala₁₀₄, decreases inhibitory activity towards both enzymes (peptide 3b). In contrast, inhibitory activity towards hCG is totally abolished by elongation through the cystein disulfide bond (see peptides 4 and 5). A dramatic rise in hLE and hCG inhibitory activity is observed when peptide 3a is elongated towards the N-terminus by the additional amino acids Asp₇₀-Ile-GIy-Tyr₇₄ peptide 3c).

Deletion of amino acid residues from both the amino and carboxy terminals leads to dramatic reduction of inhibitory activity towards hLE in comparison to the core peptide (see peptides 25 and 26 in Table 2)

Amides (CO—NH₂) of the carboxy terminal of the peptides of the invention show augmented inhibitory activity towards hLE in comparison with the core peptide 1.

N-acyl derivatives of the N-terminal have shown augmented inhibitory activity towards hCG in comparison with the core peptide 1. Examples of these acyl derivatives correspond to the formula R—X—CO— wherein R is a substituted or unsubstituted hydrocarbyl, preferably alkyl or aryl, and X is a covalent bond, O, NH or NHCO. Examples of acyl radicals are octanoyl, monomethoxysuccinyl, acetylaminocaproyl, adamantyl-NH—CO—, and more preferably, carbobenzoxy (i.e. benzyl-O—CO—), naphthyl-NH—CO—, and Fmoc (i.e. fluorenylmethyl-O—CO—).

Preferred CRP-derived peptides according to the invention are the core peptide 1; peptides obtained by substitution of His₉₅, e.g. by Phe (peptide 10); peptides obtained by elongation of peptide 1 at the amino and/or carboxy terminals and amides thereof, such as the peptides 2, 3, 4, 5, 12, and 14; and N-acyl derivatives of peptide 1, such as the peptides 16, 18, 21 and 23.

The peptides of the invention are prepared by standard methods for the synthesis of peptides. In one embodiment of the invention, the peptides are prepared as set forth in the Examples hereinbelow.

In another aspect, the present invention relates to pharmaceutical compositions comprising a peptide of the invention and a pharmaceutically acceptable carrier. The compositions are prepared by well-accepted methods for preparation of peptide-containing pharmaceutical compositions for administration in a suitable form, e.g. orally, subcutaneously, intranasal, and parenterally including intravenous, intramuscular and intraperitoneally, according to the inflammatory condition to be treated.

In a further aspect, the invention relates to a method of treatment of a chronic inflammatory condition which comprises administering to a patient in need thereof an effective amount of a peptide according to the invention. Examples of such chronic inflammatory conditions are rheumatoid arthritis, pulmonary emphysema and cystic fibrosis. The anti-inflammatory peptide is administered and dosed in accordance with good medical practice, taking into consideration the clinical condition of the patient, the site and method of administration, schedule of administration and other factors known to medical practicioners.

The invention will now be illustrated by the following non-limiting examples.

### EXAMPLES

Materials and Methods

(i) General Solid Phase Peptide Synthesis:

Peptides were prepared by conventional solid phase peptide synthesis, with ABIMED AMS-422 automated solid phase multiple peptide synthesizer (Langenfeld, Germany). The Fmoc-strategy (Fmoc=9-fluorenyl-methoxycarbonyl) was used through peptide chain assembly, following the company's commercial protocols. In each reaction vessel, 12.5 μmol of Wang resin was used which contained the first, covalently bound, corresponding N-Fmoc C-terminal amino acid (typical polymer loadings of 0.3-0.7 mmols/g resin were employed). Fmoc deprotection was achieved using duplicate flushes with 20% piperidine in dimethylformamide (DMF), typically for 10-15 min at room temperature, depending on the length of peptide and Fmoc-protected amino acid type, as given by the company's protocols.

Side chain-protecting groups were tert.-butyloxycarbonyl (t.-Boc) for Lys, diaminobutyric-acid (DAB), and Trp; trityl (Trt) for Asn, Cys, Gln, His, and (D)-His; tert.-butyl-ester (O-t-But) for Asp and Glu; tert.-butyl-ether (t-But) for Ser, Thr, and Tyr; 3-nitro-2-pyridinesulfenyl (NPYS) for Cys in the synthesis of peptides 4 and 5; and carbobenzoxy (Cbz) for the N-terminus amino acids Val and Phe in the synthesis of peptides 20 and 21, respectively.

Coupling was achieved, as a rule, using two successive reactions with 50 μmol (4 eqv.) of corresponding N-Fmoc protected amino acid, 50 μmol (4 eqv.) of PyBop reagent (benzotriazole-1-oxytris-pyrrolidino-phosphonium-hexafluoro-phosphate), and 100 μmol (μeqv.) of 4-methyl-morpholine (NMM), all dissolved in DMF, typically for 20-45 min at room temperature, depending on the length of peptide and amino acid derivative type, as given by the company's protocols.

Cleavage of the peptide from the polymer was achieved by reacting the resin with trifluoroacetic acid/H₂O/triethylsilane (TFA/H₂O/TES; 90/5/5; v/y) for 1 to 2 hours at room temperature. The crude unprotected peptides were then cooled down to 4° C., precipitated with ice-cold di-tert.-butylether (DTBE) and centrifuged for 15 min, 3000 RPM at 4° C. The pellet was washed and centrifuged 3 times with DTBE, dissolved in 30% acetonitrile in H20, and lyophilized.

All protected amino acids, coupling reagents, and polymers were obtained from Nova Biochemicals; Läufelfingen, Switzerland. Synthesis-grade solvents were obtained from Labscan; Dublin, Ireland.

(ii) Reversed-phase High Performance Liquid Chromatography (RP-HPLC):

Synthetic peptides were purified by using a prepacked LiChroCart RP-18 column (250×10 mm, 7 μm bead size), employing a binary gradient formed from 0.1% TFA in H₂O (solution A) and 0.1% TFA with 25% H₂O in acetonitrile (solution B), eluted at t=0 min B=5% t=5 min B=5% t=60 min B=70% (flow-rate 5 mL/min). Analytical RP-HPLC was performed using a prepacked Lichrospher-100 RP-18 column (4×250 mm, 5 μm bead size) using the same buffer system (flow-rate 0.8 mL/min). All peptide separations were performed using a Spectra-Physics SP8800 liquid chromatography system equipped with an Applied Biosystems 757 variable wave-length absorbence detector. The column effluents were monitored by UV absorbence at 220 nm, and chromatograms were recorded on a Chrome-Jet integrator. Following HPLC purification, the lyophilized peptides (generally >90% pure for crude samples after synthesis as described below) were purified to >97%. All solvents and HPLC columns were obtained from Merck; Darmstadt, Germany.

(iii) Amino Acid Composition Analysis:

Purified peptide solutions were roto-evaporated (≈40 μg of peptide in 40 μL solution with 5 μg of norleucine as an un-natural amino acid internal standard), hydrolyzed in 6 N HCl at 110° C. for 22 hours under vacuum and analyzed with a Dionex amino acid analyzer. This quantification was used as a basis for determination of the total yield of peptide. Several of the peptides synthesized were analyzed by Liquid Secondary-ion Mass-spectrometry which confirmed their expected (M+H)⁺, protonated molecular ions.

(iv) Isolation of hLE and hCG:

The isolation of neutrophilic enzymes was based on the two-step aprotinin-sepharose affinity chromatography and carboxymethyl-cellulose (CMC) ion exchange chromatography (Heck et al., 1985). Neutrophils (1.4 billion) were isolated from whole blood obtained from a single healthy laboratory donor by dextran sedimentation and Ficolllhypaque gradient centrifugations as described elsewhere (Metcalf et al., 1986). The enzymatic activity was assayed with MeOSuc-AAPV-NA for hLE determination and Suc-AAPF-NA for hCG determination (both in 100 mM Hepes buffer, pH 7.4, containing 0.05% of the anionic detergent Brij-35). The activities of the individual enzymatic fractions were 100% free from cross-contamination. The step-wise elution profile on the CMC column with a long 0.45 M NaCl elution step (20 column volumes) afforded the effective separation between the two enzymes. The fractions containing hLE and hCG were dialyzed each against 0.1% pyridinium acetate, pH 5.3, divided into 20 aliquots, lyophilized, and stored at −20° C. until use. By the initial rates of reactions and the known values of Kcat (hLE=54 μM, hCG=2900 μM) and Kₘ (hLE=B13.3 sec.⁻¹, hCG=3.1 sec.⁻¹), the amount of enzyme was estimated to be approximately 15 μg/aliquot for hLE and 12 μg/aliquot for hCG, such values being confirmed by active site titration with ccI-PI and ACT.

(v) Inhibition Experiments With hLE:

Peptides were dissolved in 100 mM Hepes buffer pH 7.4 containing 0.1% Brij-35 with 10% DMSO to yield 600 μM solutions, which were used to make further dilutions with the same buffer, and 80 μL aliquots were added in duplicates to 96-well plates. The substrate, 600 or 900 μM MeOSuc-AAPV-NA in the same buffer with 5% DMSO, was added to each well in addition to the blank wells, and the plate was placed in the plate reader equilibrated to 37° C. (Dynateck MR-6000). Lyophilized aliquots of hLE were dissolved in 1600 μL of 100 mM Hepes buffer without DMSO, and 80 μL of the enzyme solution was added to the peptides and substrate to initiate the reaction. The kinetics program read the plate at 405 nm every 2 min for 20 min (with a 3 sec shaking period between readings), and plotted the results as well as the average of each duplicate. The final volume was 240 μL containing: 5% DMSO, 1-200 μM of peptide, 200 μM substrate, and 0.75 μg (about 25 picomol) enzyme.

(vi) Inhibition Experiments With hCG:

Similar conditions to hLE inhibition experiments ere used except the substrate: 80 μL of 1.8 mM Suc-AAPF-NA. The enzyme was issolved in 800 μL buffer, and the reaction was monitored every 6 min for 1 hour. The final volume was 240 μL containing: 5% DMSO, 1-500 μM peptide, 600 μM substrate, and 1.2 μg enzyme (about 40 picomol).

(vii) Degradation Profiles of Peptides by RP-HPLC:

Several active peptide inhibitors were dissolved in calcium- and magnesium-free phosphate-buffered saline (PBS), 125 μg/250 μL, mixed with 0.25 aliquots of hLE or hCG in 250 μL PBS and incubated at 37° C. Periodically (1,3,8, and 24 hour), 100 μL samples were removed from the reaction vessel. The samples were diluted with 150 μL of 0.1% TFA, frozen with liquid nitrogen, and stored at −20° C. prior to HPLC analysis.

(viii) Calculations:

For hLE, V is determined by fitting a linear equation to the first 6 time-points (10 min) of the kinetics data using the least squares method. Without exception, all R² factors were >0.998. Several inhibitor concentration (250, 375, and 500 μM) in duplicates and two control wells were used to fit a linear equation to graphs of Vo/Vᵢ-1 vs. [I] for each inhibitor using the least squares method (8 data points for each inhibitor). From calculating the error in the slope of the equation, the relative error for Kᵢ was deduced:

Kᵢ={slope*(1+[S]/Kₘ)}⁻¹ because Kᵢ=[I]*{(1+[S]/Kₘ)*(Vo/Vᵢ₋1)}⁻¹.

For hCG, V is determined by fitting a quadratic equation to the total kinetic data (60 min), using the least squares method and calculating V at t=0. Without exception, all R² factors were >0.996. Two inhibitor concentrations (250 and 500 μM) in duplicate and two control wells were used for each inhibitor, and in a similar fashion to hLE, Kᵢ was deduced (6 data points for each inhibitor).

### Example 1

### Synthesis of Core Peptide 1 and Other Peptides

The sequence of the peptides 1-23 according to the invention and the inhibition constants (Kᵢ) of human hLE and human hCG are shown in Table 1. The sequences of the comparison peptides 24-30 and the hLE Kᵢ are shown in Table 2. The amino acid analysis of peptides 1-30 is shown in Table 3.

TABLE 1
CRP-derived peptides of the invention and inhibition constants (Ki) of
human leukocyte elastase (hLE) and human cathepsin G (hCG).

| Peptide | Sequence | hLE Ki (μM) | hCG Ki (μM) |
|---|---|---|---|
| 1 | Val₈₉-Thr-Val-Ala-Pro-Val-His-Ile₉₆ | 120 ± 15 | 1400 ± 200 |
| 2 | Gly₇₉-Ser-Glu-Ile-Leu-Phe-Glu-Val-Pro-Glu-Val- | 50 ± 5 | 1200 ± 200 |
| | Thr-Val-Ala-Pro-Val-His-Ile₉₆ | | |
| 3 | Ser₇₄-Phe-Thr-Val-Gly-Gly-Ser-Glu-Ile-Leu-Phe- | 27 ± 3 | 500 ± 100 |
| | Glu-Val-Pro-Glu-Val-Thr-Val-Ala-Pro-Val-His- | | |
| | Ile₉₆ | | |
| 3a | Ser₇₄-Phe-Thr-Val-Gly-Gly-Ser-Glu-Ile-Leu-Phe- | 20 ± 3 | 180 ± 30 |
| | Glu-Val-Pro-Glu-Val-Thr-Val-Ala-Pro-Val-His- | | |
| | Ile-Cys-Thr₉₈ | | |
| 3b | Ser₇₄-Phe-Thr-Val-Gly-Gly-Ser-Glu-Ile-Leu-Phe- | 22 ± 4 | 200 ± 30 |
| | Glu-Val-Pro-Glu-Val-Thr-Val-Ala-Pro-Val-His- | | |
| | Ile-Cys-Thr-Ser-Trp-Glu-Ser-Ala₁₀₃ | | |
| 3c | Asp₇₀-Ile-Gly-Tyr-Ser-Phe-Thr-Val-Gly-Gly-Ser- | 4.0 ± 0.6 | 22 ± 3.0 |
| | Glu-Ile-Leu-Phe-Glu-Val-Pro-Glu-Val-Thr-Val- | | |
| | Ala-Pro-Val-His-Ile-Cys-Thr₉₈ | | |
| 4 | Val₈₉-Thr-Val-Ala-Pro-Val-His-Ile-Cys₉₇- | 85 ± 5 | N.I. |
| | Cys₃₆-His-Leu-Phe₃₉ | | |
| 5 | Gly₇₉-Ser-Glu-Ile-Leu-Phe-Glu-Val-Pro-Glu-Val- | 55 ± 5 | N.I. |
| | Thr-Val-Ala-Pro-Val-His-Ile-Cys₉₇-Cys₃₆-His- | | |
| | Leu-Phe₃₉ | | |
| 6 | Val₈₉-Thr-Val-Ala-Pro-Val-(D)His-Ile₉₆ | 450 ± 45 | N.I. |
| 7 | Val₈₉-Thr-Val-Ala-Pro-(D)Val-His-Ile₉ | 330 ± 130 | N.I. |
| 8 | Val₈₉-Thr-Val-Ala-Pro-(D)Val-(D)His-Ile₉₆ | 490 ± 50 | W.I. |
| 9 | Val₈₉-Thr-Val-Ala-Pro-Val-Ser-Ile₉₆ | 200 ± 20 | N.I. |
| 10 | Val₈₉-Thr-Val-Ala-Pro-Val-Phe-Ile₉₆ | 110 ± 25 | 1.0 ± 0.2 |
| 12 | Val₈₉-Thr-Val-Ala-Pro-Val-His-Ile₉₆-Pro-NH₂ | 70 ± 5 | W.I. |
| 13 | Val₈₉-Thr-Val-Ala-Pro-Phe-His-Ile₉₆-Pro-NH₂ | 180 ± 30 | 900 ± 200 |
| 14 | Val₈₉-Thr-Val-Ala-Pro-Val-His-Ile₉₆-Pro-Pro- | 85 ± 10 | W.I. |
| | NH₂ | | |
| 15 | MeOSuc-Val₈₉-Thr-Val-Ala-Pro-Val-His-Ile₉₆ | 160 ± 30 | W.I. |
| 16 | MeOSuc-Phe-Val₈₉-Thr-Val-Ala-Pro-Val-His- | 100 ± 10 | 100 ± 200 |
| | Ile₉₆ | | |
| 17 | Adamantyl-NH-CO-Val₈₉-Thr-Val-Ala-Pro-Val- | 130 ± 15 | W.I. |
| | His-Ile₉₆ | | |
| 18 | Naphtyl-NH-CO-Val₈₉-Thr-Val-Ala-Pro-Val- | 240 ± 35 | 300 ± 40 |
| | His-Ile₉₆ | | |
| 19 | Octanoyl-Val₈₉-Thr-Val-Ala-Pro-Val-His-Ile₉₆ | 280 ± 25 | W.I. |
| 20 | CBz-Val₈₉-Thr-Val-Ala-Pro-Val-His-Ile₉₆ | 165 ± 35 | N.I. |
| 21 | CBz-Phe-Val₈₉-Thr-Val-Ala-Pro-Val-His-Ile₉₆ | 380 ± 115 | 430 ± 60 |
| 22 | Acetyl-aminocaproyl-Val₈₉-Thr-Val-Ala-Pro- | 210 ± 30 | W.I. |
| | Val-His-Ile₉₆ | | |
| 23 | Fmoc-Val₈₉-Thr-Val-Ala-Pro-Val-His-Ile₉₆ | 1000 ± 100 | 280 ± 30 |

W.I.; weak inhibition. N.I.; no detected inhibition. Subscript numbers relate to the position of the peptide within the primary sequence of CRP and bold letters denote amino acid or organic modifications. MeOSuc is monomethoxy-succinyl, CBz is the carbobenzoxy protecting group, Acetyl-aminocaproic is 6-acetylamino-N-hexanoyl and Fmoc is 9-fluorenylmethoxycarbonyl.

TABLE 2
Comparison peptides and inhibition constants of hLE

| Peptide | Sequence | hLE Ki (μM) |
|---|---|---|
| 24 | Leu-Glu-Ala-Ile-Pro-Met-Ser-Ile (from α₁-PI) | 350 ± 70 |
| 25 | Val₈₉-Thr-Val-Ala-Pro-Val₉₄ | 900 ± 300 |
| 26 | Val₉₁-Ala-Pro-Val-His-Ile₉₆ | 1000 ± 250 |
| 27 | Val₈₉-Thr-Val-Ala-(D)Pro-Val-His-Ile₉₆ | 560 ± 170 |
| 28 | Val₈₉-Thr-Val-Ala-(D)Pro-(D)Val-(D)His-Ile₉₆ | 2300 ± 300 |
| 29 | Val₈₉-Thr-Val-Ala-Pro-Val-DAB-Ile₉₆ | 500 ± 170 |
| 30 | Val₈₉-Thr-Val-Ala-Sarcosine-Val-His-Ile₉₆ | 1400 ± 500 |

Subscript numbers relate to the position of the peptide within the primary sequence of CRP and bold letters denote amino acid modifications. Sarcosine is N-methyl glycine and DAB is 1,4-(L)diaminobutyric acid.

TABLE 3
Amino acid analysis ratios and HPLC retention time data for peptides 1-30

Peptide 1. AAA: Thr 1.01(1), Pro 1.01(1), Ala 1.01(1), Val 3.10(3), Ile 1.01(1), His
0.99(1). HPLC: R.T. 37.9 min.
Peptide 2. AAA: Thr 0.85(1), Ser 0.96(1), Glu 3.13(3), Pro 2.18(2), Gly 0.96(1), Ala
1.02(1), Val 4.08(4), Ile 2.00(2), Leu 1.01(1), Phe 0.98(1), His 0.96(1). HPLC: R.T.51.3
min.
Peptide 3. AAA: Thr 1.97(2), Ser 1.98(2), Glu 2.64(3), Pro 1.61(2), Gly 2.02(2), Ala
1.04(1), Val 5.11(5), Ile 1.98(2), Leu 1.00(1), Phe 1.99(2). HPLC: R.T. 54.1 min.
Peptide 3a. AAA: Glu 3.0 (3), Ser 1.9 (2), His 1.0 (1), Gly 2.0 (2), Thr 3.0 (3), Ala 1.0 (1),
Cys 0.9 (1), Val 5.0 (5), Ile 2.0 (2), Phe 2.0 (2), Leu 1.1 (1), Pro 2.0 (2). HPLC R.T. = 31.1.
Peptide 3b. AAA: Glu 4.0 (4), Ser 3.9 (3), His 1.0 (1), Gly 2.0 (2), Thr 3.0 (3), Ala 1.9 (1),
Cys 1.0 (1), Val 5.0 (5), Ile 2.1 (2), Phe 2.0 (2), Leu 0.9 (1), Pro 2.0 (2). HPLC R.T. = 32.2
Peptide 3c. AAA: Asp 1.0 (1), Glu 3.1 (3), Ser 2.0 (2), His 1.0 (1), Gly 3.0 (3), Thr 2.9 (3),
Ala 1.0 (1), Tyr 0.9 (1), Cys 0.9 (1), Val 5.0 (5), Ile 3.0 (3), Phe 2.0 (2), Leu 1.0 (1), Pro
2.0 (2). HPLC R.T. = 32.3
Peptide 4. AAA: Thr 1.12(1), Pro 0.97(1), Cys 0.98(2), Val 3.46(3), Ile 1.00(1), Leu
1.43(1), Phe 1.36(1) His 1.98(2). HPLC: R.T. 44.8 min.
Peptide 5. AAA: Thr 0.92(1), Ser 0.91(1), Glu 3.00(3), Pro 1.87(2), Gly 0.98(1), Ala
1.00(1), Cys 0.90(2), Val 4.06(4), Ile 1.88(2), Leu 2.21(2), Phe 2.20(2) His 2.05(1). HPLC
R.T. = 51.9 min.
Peptide 6. AAA: Val, 2.99(3); Thr, 0.97(1); Ala, 1.01(1); Pro, 1.00(1); His, 0.98(1); Ile,
1.00(1). HPLC R.T. = 26.7 min.
Peptide 7. AAA: Val, 2.98(3); Thr, 0.95(1); Ala, 1.01(1); Pro, 1.00(1); His, 0.97(1); Ile,
0.98(1). HPLC R.T. = 24.6 min.
Peptide 8. AAA: Val, 3.00(3); Thr, 0.96(1); Ala, 1.02(1); Pro, 1.05(1); His, 1.00(1); Ile,
1.04(1). HPLC R.T. = 26.7 min.
Peptide 9. AAA: Val, 3.15(3); Thr, 0.93(1); Ala, 1.01(1); Pro, 1.01(1); Ser, 0.95(1); Ile,
1.00(1). HPLC R.T. = 27.0 min.
Peptide 10. AAA: Val, 3.03(3); Thr, 0.96(1); Ala, 1.03(1); Pro, 1.05(1); Phe, 0.98(1);
Ile, 1.01(1). HPLC R.T. = 27.3 min.
Peptide 11. AAA: Val, 2.97(3); Thr, 0.94(1); Ala, 1.03(1); Pro, 1.09(1); His, 0.94(1);
Ile, 0,97(1). HPLC R.T. = 24,5 min.
Peptide 12. AAA: Val, 2.98(3); Thr, 0.95(1); Ala, 1.03(1); Pro, 2.04(2); His, 0.96(1);
Ile, 0.99(1). HPLC R.T. = 25.3 min.
Peptide 13. AAA: Val, 1.99(2); Thr, 0.96(1); Ala, 1.00(1); Pro, 2.03(2); His, 0.98(1);
Ile, 0.94(1); Phe, 1.01(1). HPLC R.T. = 30.0 min.
Peptide 14. AAA: Val, 2.96(3); Thr, 0.95(1); Ala, 1.04(1); Pro, 3.13(3); His, 0.94(1);
Ile, 0.95(1). HPLC R.T. = 24.8 min.
Peptide 15. AAA: Val, 3.01(3); Thr, 0.95(1); Ala, 1.03(1); Pro, 0.98(1); His, 1.00(1);
Ile, 1.01(1). HPLC R.T. = 20.8 min.
Peptide 16. AAA: Val, 3.02(3); Thr, 0.93(1); Ala, 1.30(1); Pro, 1.02(1); His, 1.01(1);
Ile, 1.00(1). HPLC R.T. = 25.3 min.
Peptide 17. AAA: Val, 2.63(3); Thr, 1.09(1); Ala, 0.99(1); Pro, 1.20(1); His, 1.16(1);
Ile, 1.11(1). HPLC R.T. = 29.8 min.
Peptide 18. AAA: Val, 2.92(3); Thr, 0.97(1); Ala, 0.91(1); Pro, 0.97(1); His, 1.02(1);
Ile, 1.00(1). HPLC R.T. = 29.1 min.
Peptide 19. AAA: Val, 3.03(3); Thr, 0.96(1); Ala, 0.89(l); Pro, 0.90(1); His, 1.03(1);
Ile, 0.97(1). HPLC R.T. = 30.1 min.
Peptide 20. AAA: Val, 3.02(3); Thr, 1.16(1); Ala, 1.29(1); Pro, 1.04(1); His, 1.02(1);
Ile, 0.98(1). HPLC R.T. = 29.4 min.
Peptide 21. AAA: Val, 3.02(3); Thr, 0.95(1); Ala, 0.88(1); Pro, 0.97(1); His, 1.02(1);
Ile, 1.00(1). HPLC R.T. = 30.4 min.
Peptide 22. AAA: Val, 3.01(3); Thr, 0.94(1); Ala, 1.09(1); Pro, 1.01(1); His, 1.04(1);
Ile, 1.00(1). HPLC R.T. = 29.1 min.
Peptide 23. AAA: Val. 2.90(3); Thr, 1.00(1); Ala, 1.00(1); Pro, 1.02(1); His, 1.02(1);
Ile, 0.98(1). HPLC R.T. = 31.2 min.
Peptide 24. AAA: Ser 1.00(1), Glu 0.97(1), Pro 0.94(1), Ala 1.03(1), Met 1.02(1), Ile
2.13(2), Leu 1.06(1). HPLC: R.T. = 44.9 min.
Peptide 25. AAA: Thr 1.00(1), Pro 0.99(1), Ala 1.00(1), Val 2.08(2). HPLC: R.T.30.6
min.
Peptide 26. AAA: Pro 1.00(1), Ala 1.01(1), Val 2.03(2), Ile 1.00(1), His 0.98(1). HPLC:
R.T. 34.5 min.
Peptide 27. AAA: Val, 3.00(3); Thr, 1.01(1); Ala, 1.03(1); Pro, 1.02(1); His, 0.95(1);
Ile, 0.98(1). HPLC R.T. = 29.3 min.
Peptide 28. AAA: Val, 2.97(3); Thr, 0.96(1); Ala, 1.02(1); Pro, 1.04(1); His, 0.97(1);
Ile, 1.00(1). HPLC R.T. = 27.8 min.
Peptide 29. AAA: Val, 3.09(3); Thr, 0.96(1); Ala, 1.01(1); Pro, 1.01(1); DAB, 1.03(1);
Ile, 1.04(1). HPLC R.T. = 26.6 min.
Peptide 30. AAA: Val, 3.03(3); Thr, 0.95(1); Ala, 1.03(1); Sar, 0.96(1); His, 1.03(1);
Ile, 1.01(1). HPLC R.T. = 27.1 min.

HPLC retention times (RT) for peptides 6-23 and 27-30 are given for the following gradient: t = 0 min. B = 5%, t = 5 min. B = 5%, t = 55 min. B = 100 %.
HPLC retention times for peptides 1-5 and 24-26 are given for the following gradient: t = 0 min. B = 5%, t = 5 min. B = 5%, t = 60 min. B = 70 %.
HPLC retention times for peptides 3a-3c are given for the following gradient: t = 0 min. B = 10%, t = 2 min. B = 10%, t = 50 min. B = 90%.

### Example 1.1

### Synthesis of Core Peptide 1

In the synthesis of peptide 1, H-Val-Thr-Val-Ala-Pro-Val-His-Ile-OH,
the standard Fmoc protocol was used as follows:

| Peptide elongation cycle: | |
|---|---|
| Step 1. DMF wash | ×6 |
| Step 2. Deprotection: 20% piperidine in DMF | ×2 |
| Step 3. DMF wash | ×6 |
| Step 4. Derivative coupling. | ×2 |
| At the end of synthesis: | |
| Step 1. DMF wash | ×6 |
| Step 2. Deprotection: 20% piperidine in DMF | ×2 |
| Step 3. DMF wash | ×6 |
| Step 4. CH₂Cl₂ wash | ×6 |

Deprotection, coupling and wash times and volumes, were calculated by the ABIMED computer program. The resulting lyophilized crude peptide was purified by preparative HPLC to yield approx. 12 mg of lyophilized peptide (white powder), above 99% pure, as determined by its analytical RP-HPLC peak eluting at 37.9 min. Amino acid analysis confirmed the expected sequence, purity, and yield of purified peptide (see Table 3 above).

### Example 1.2

### Synthesis of Peptides 2, 3, 3a-3c, 6-10 and 24-30

Peptides 2, 3, 3a-3c, 6-10, and 24-30 were prepared by the standard Fmoc protocol in a similar fashion as described in 1.1 above.

### Example 1.3

### Synthesis of Disulfide Bridged Peptides 4 and 5

Synthesis of peptides 4 and 5 was carried out according to Scheme 1 hereinafter: the unsymmetrical bridging (oxidation) of two cysteine bonds was performed using a polymer-bound peptidic fragment containing the sulfur-bound NPYS protecting group, which reacts rapidly with the exposed S-H of a pre-purified peptide in solution phase, thus combining the two peptidic fragments.

In the synthesis of Peptide 4, the peptide H-Val-Thr-Val-Ala-Pro-Val-His-Ile-Cys-OH was prepared using the standard Fmoc protocol, and purified to above 98% by preparative HPLC as described above. The lyophilized peptide (20 mg≈20 μmol) was dissolved in 1 mL N-methyl-pyrrolidone (NMP), and added to 45 mg (≈18 μmol) of Fmoc-Cys(NPYS)-Leu-His(Trt)-Phe-Polymer suspended in 1 mL NMP. The combined solution was titrated to apparent basic pH8 with 5% triethylamine in NMP. The reaction mixture was gently rocked for 1 hour at room temperature, and the liberated nitroaromatic compound was observed to yield a dark green color. The polymer was washed thoroughly with NMP followed by CH₂Cl₂. The Fmoc group was removed, and the peptide was cleaved from the polymer as described above. The highest yield was obtained by using non-aqueous NMP at apparent pH 8-8.5 as opposed to reactions carried out in mixed organic aqueous solutions such as DMF/H₂O or CH₃CN/H₂O.

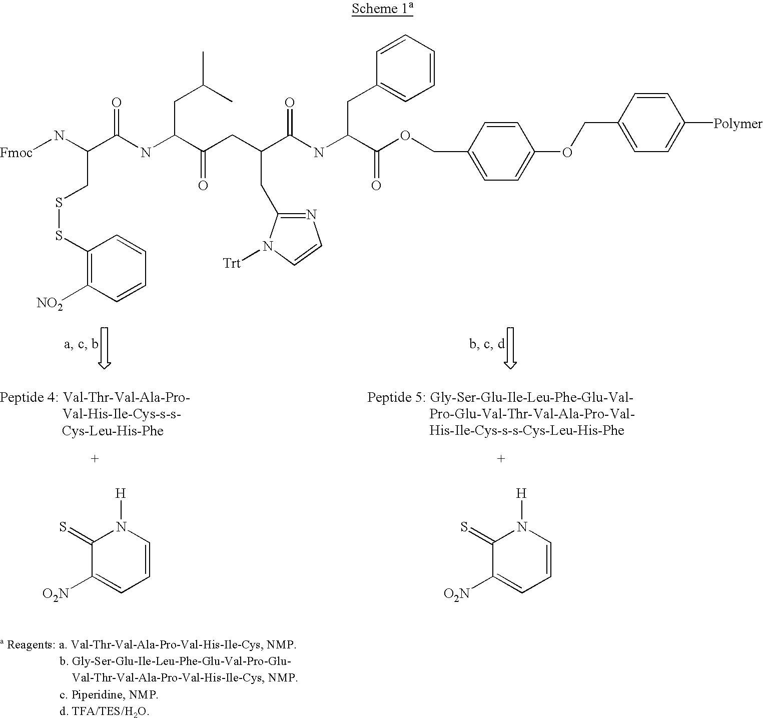

The analytical RP-HPLC chrormatogram of the product was observed to contain a single peak eluting at a longer retention time (51.4 min.) as compared with the precursor reactants. To confirm the integrity of the disulfide bond, 50 μg of peptide was treated with 100 μL of I M aqueous 1,4-dithiothreitol (1 hour at pH 8 using 5% ammonia) to yield the two original HS free-containing fragments exclusively as determined by RP-HPLC co-elution at 37.8 min. and 39.8 min.

In the synthesis of Peptide 5, the peptide H-Gly-Ser-Glu-Ile-Leu-Phe-Glu-Val-Pro-Glu-Val-Thr-Val-Ala-Pro-Val-His-Ile-Cys-OH was prepared and purified to above 97% by preparative RP-HPLC. The lyophilized peptide (30 mg≈25 μmol) was dissolved in 1.5 mL NMP, and added to 35 mg (≈14 μmol) of Fmoc-Cys(NPYS)-Leu-His(Trt)-Phe-Polymer suspended in 1 mL NMP and reacted in a similar fashion to peptide 4 as described above with a yield of 75%. The analytical HPLC chromatogram of the product contained a single peak eluting at a longer retention time (54.1 min) as compared with the parent reactants. Reducing 50 μg of peptide with 50 μL of 1 M aqueous 1,4-dithiothreitol (1 hour at pH 8 using 5% ammonia) yields the two original fragments exclusively as determined by RP-HPLC co-elution at 51.9 min. and 39.8 min.

### Example 1.4

### Synthesis of Peptides 11-14

In the synthesis of peptide 11, H-Val-Thr-Val-Ala-Pro-Val-His-Ile-NH₂, the standard resin was replaced with 12.5 μmols of rink amide solid support [4-2′(4′-dimethoxyphenyl-Fmoc-aminomethyl)-phenoxy-resin] which does not contain the first amino acid. Peptide synthesis was followed in an identical fashion as described in 1.1 above, and upon cleavage from the polymer, the carboxy terminus amidated form of the peptide is obtained. The resulting lyophylized crude peptide was purified by preparative RP-HPLC to yield approx. 8 mg of lyophilized peptide (white powder), above 98% pure, as determined by its analytical RP-HPLC peak eluting at 24.5 min.

Peptides 12, 13, and 14 were prepared in an identical fashion.

### Example 1.5

### Synthesis of Peptides 15-16

In the synthesis of peptide 15, CH₃OCO(CH₂)₂CO-Val-Thr-Val-Ala-Pro-Val-His-Ile-OH, monomethyl-succinlc-acid was coupled to the exposed N-terminus of H-Val-Thr(t.-But)-Val-Ala-Pro-Val-His(Trt)-Ile-Polymer as the final step of solid phase peptide synthesis. Mono-methyl-succinic-acid (100 μmols, 8 μeqv.), PyBOP (100 μmols), and NMM (200 μmols) were dissolved in 2 mL NMP and added to the resin bound peptide (12.5 μmols) for 1 hour at room temperature followed by extensive flushing with NMP and CH₂Cl₂. The peptide was then cleaved from the polymer and purified by preparative HPLC as described above (sections i and ii of Materials and Methods), to yield approx. 10 mg of lyophilized peptide (white powder), above 98% pure, as determined by its analytical RP-HPLC peak eluting at 20.8 min.

Peptide 16, CH₃OCO(CH₂)₂CO-Phe-Val-Thr-Val-Ala-ProVal-His-Ile-OH, was prepared in an identical fashion using H-Phe-Val-Thr-Val-Ala-Pro-Val-His-Ile-Polymer as the polymer bound peptide.

### Example 1.6

### Synthesis of Peptides 17-18

In the synthesis of peptide 17, 1-adamantyl-NH—CO-Val-Thr-Val-Ala-Pro-Val-His-Ile-OH, 1-adamantyl isocyanate (100 μmols, 8 μeqv.) was coupled to the N-terminus of H-Val-Thr(t-But)-Val-Ala-Pro-Val-His(Trt)-Ile-Polymer (12.5 μmols), as the final stage of solid phase peptide synthesis. The isocyanate compound was allowed to react (without PyBOP or NMM) in 2 mL NMP for 4 hours at room temperature followed by extensive flushing with NMP and CH₂Cl₂. The peptide was then cleaved from the polymer and purified by preparative BPLC as described above (sections i and ii of Materials and Methods), to yield approx. 10 mg of lyophilized product, above 97% pure, as determined by the analytical RP-HPLC peak eluting at 29.8 min.

In a similar way, Peptide 18, a-naphtyl-NH-CO-Val-Thr-Val-Ala-Pro-Val-His-Ile-OH, was synthesized using a-naphtyl isocyanate (100 μmols, 8 μeqv.).

### Example 1.7

### Synthesis of Peptide 19

In the synthesis of peptide 19, CH₃(CH₂)₆CO-Val-Thr-Val-Ala-Pro-Val-His-Ile-OH, normal-octanoic acid (100 μmols, 8 μeqv.) was coupled to the N-terminus of H-Val-Thr(t-But)-Val-Ala-Pro-Val-His(Trt)-Ile-Polymer (12.5 μmols) as the final step of solid phase peptide synthesis, using PyBOP (100 μmols) and NMM (200 μmols) in 2 mL NMP for 1 hour at room temperature followed by extensive flushing with NMP and CH₂Cl₂. The peptide was then cleaved from the polymer and purified by preparative RP-HPLC as described above (sections i and ii of Materials and Methods), to yield approx. 10 mg of lyophilized peptide (white powder), above 98% pure, as determined by its analytical RP-HPLC peak eluting at 30.1 min.

### Example 1.8

### Synthesis of Peptides 20 and 21

In the synthesis of peptides 20 and 21, carbobenzoxy N-terminus protected amino acids were utilized (50 μmols CBz-Val and 50 μmols CBz-Phe, respectively) as the last amino acid coupling, using identical coupling conditions and 12.5 μmols of polymer, as described in 1.1 above. The carbobenzoxy moiety is stable under peptide-polymer cleavage conditions, which yields the N-terminus derived peptides: CBz-Val-Thr-Val-Ala-Pro-Val-His-Ile-OH and CBz-Phe-Val-Thr-Val-Ala-Pro-Val-His-Ile-OH, respectively.

### Example 1.9

### Synthesis of Peptide 22

In the synthesis of peptide 22, CH₃CONH(CH₂)₅CO-Val-Thr-Val-Ala-Pro-Val-His-Ile-OH, N-acetyl-amino-caproic acid (100 μmols, 8 μeqv.) was coupled to the N-terminus of H-Val-Thr(t-But)-Val-Ala-Pro-Val-His(Trt)-Ile-Polymer (12.5 μmols), as the final step of solid phase peptide synthesis, using PyBOP (100 μmols) and NMM (200 μmols) in 2 mL NMP for 1 hour at room temperature followed by extensive flushing with NMP and CH₂Cl₂. The peptide was then cleaved from the polymer and purified by reparative RP-HPLC as described above, to yield approx. 12 mg of lyophilized peptide (white powder), above 97% pure, as determined by its analytical RP-HPLC peak eluting at 29.1 min.

### Example 1.10

### Synthesis of Peptide 23

In the synthesis of peptide 23, the final step of Fmoc deprotection of the peptide was omitted. The Fmoc moiety is stable under peptide-polymer cleavage and side-chain deprotection conditions, yielding the N-terminus derived peptide: Fmoc-Val-Thr-Val-Ala-Pro-Val-His-Ile-OH.

### Example 2

### In-Vitro Inhibition of hLE by Peptides 1-30

The hLE inhibitory capability of CRP-derived peptides was evaluated by inhibiting the enzymatic cleavage of MeOSuc-AAPV-NA as described in Materials and Methods (section v). The results are shown in Table I for peptides according to the invention and in Table 2 for comparison peptides.

The CRP-derived core octapeptide 1, Val₈₉-Thr-Val-Ala-Pro-Val-His-Ile₉₆, is shown to be a more potent inhibitor of hLE than the α₁-PI-derived core octapeptide 24. The inhibitory activity of a peptide with the sequence Val-Ala-Pro-Val is minute (data not shown). The inhibitory activity of the core peptide drops drastically with removal of the residues His-Ile from the carboxy terminus (peptide 25) or Val-Thr from the amino terminus (peptide 26). Replacing His₉₅ with Ser (peptide 9) yields inhibitory activity similar to the original peptide while replacement by Phe (peptide 10) even increases the inhibitory activity.

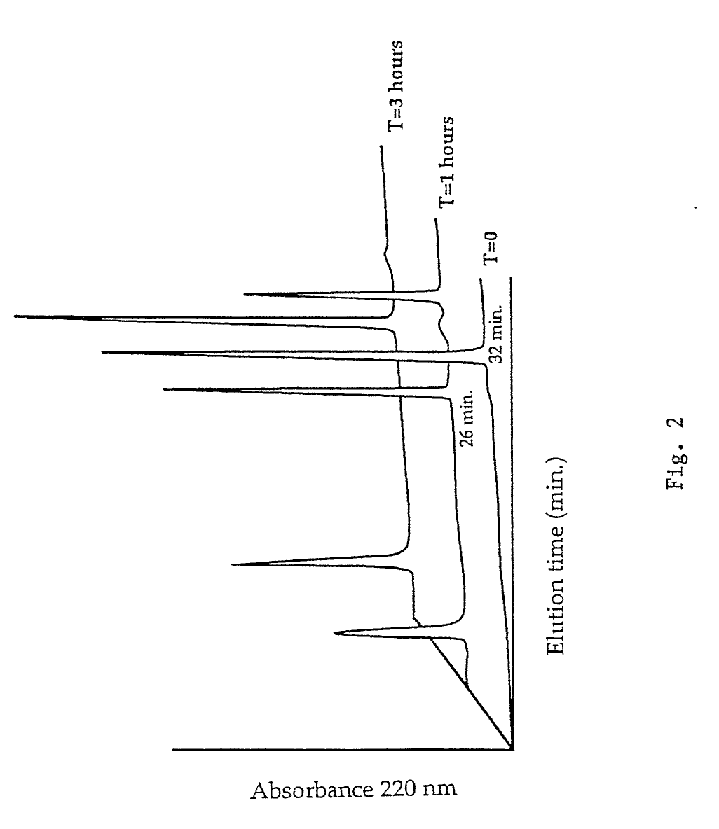

More insight into the mechanism of inhibition is provided by HPLC time-course elution profiles of peptide 1, incubated with hLE in PBS (FIG. 2). The peptide is cleaved as predicted exclusively at the Val-His bond generating the expected two fragments, identified by co-elution with peptide 25. The other bonds in peptide 1 are cleaved only after several days of incubation with the enzyme. No observable cleavage products are detected in the incubation of hLE with peptides 6, 7 and 8 during the same time-scale (hours), indicating that the D analogues are effective in resisting degradation by the enzyme.

The extended sequence of peptide 1 at the amino terminal (peptides 2,3) increases the inhibitory capacity on an equimolar basis. The C-terminus addition of Cys₉₇-Thr₉₈ to peptide 3 combined with the addition of N-terminus amino acids Asp₇₀-Ile-Gly-Tyr₇₄ (peptide 3c) increases dramatically the inhibitory activity of Ki=4 μM towards hLE. Extension of the sequence at the carboxy terminus (peptide 4) via the disulfide bridge increases to a lesser extent the inhibitory capability.

From the carboxy terminal modifications of the core peptide 1 (peptides 11-14), -Pro-NH₂ appears to be the most beneficial modification (peptide 12), increasing substantially the hLE inhibitory capability.

From the amino terminal modifications of the core peptide 1 (peptides 15-23), methoxysuccinyl-Phe-appears to be the most beneficial modification (peptide 16), increasing substantially the hLE inhibitory capability.

In contrast, replacement of His₉₅ by the charged moiety of diaminobutyric acid (DAB-peptide 29) reduces dramatically the inhibitory capability. When Pro was replaced by sarcosine (peptide 30), inhibitory capability was almost completely lost, stressing the structural importance of proline in these CRP-derived peptides.

The hLE inhibitory capability of several peptides derived from various regions within the sequence of CRP were evaluated. No significant inhibition was observed for any of the following peptides: Asn₁₆₀-Met-Trp-Asp-Phe-Val₁₆₅, Ser₁₈-Tyr-Val-Ser-Leu-Lys₂₃, Asp₇₀-Ile-Gly-Tyr-Ser₇₄, Val₁₅₃-Gly-Asp-Ile-Gly-Asn-Val₁₅₉, Asp₁₁₂-Gly-Lys-Pro-Arg-Val-Arg-Lys₁₁₉, Gln₂₀₃-Leu-Trp-Pro₂₀₆, Thr₂₀₀-Lys-Pro-Gln-Leu-Trp-Pro₂₀₆, Thr₇₆-Val-Gly-Gly-Ser₈₀ and Phe₈₄-Glu-Val-Pro-Glu-Val-Thr₉₀.

### Example 3

### In-Vitro Inhibition of hCG by Peptides 1-23

The inhibitory capability of several CRP-derived peptides was evaluated by inhibiting the enzymatic conversion of Suc-AAPF-NA (as described in Materials and Methods (vi) above). The results are shown in Table 1 above. Peptide 1 is slightly inhibitory with a dramatic rise in the inhibitory capacity as the amino terminal is elongated (peptide 3c is the most prominent). In contrast, peptides 4 and 5 are completely inactive, indicating a poor fit of the disulfide portion of the peptide within the enzyme's subsites.

The most prominent modifications of the core peptide 1 that increase inhibitory activity towards hCG are the aromatic acyl derivatives 18, 21 and 23.

### REFERENCES

1. Ballue, S. P. & Kushner, I. (1992) C-reactive protein and the acute phase response. Advances in Internal Medicine, 37, 313-336.

2. Bode, B., Meyer, E. & Powers, C. J. (1989) Human leukocyte elastase and porcine pancreatic elastase: X-ray crystal structures, mechanism, substrate specifity and mechanism based inhibitors. Biochemistry, 28(5), 1951-1963.

3. Edwards, P. D., Bernstein, P. R. (1994) Synthetic inhibitors of Elastase. Med. Res. Rev. 14, 127-194 and references cited therein.

4. Finke, P. E. et al., (1995) Orally active P-lactam inhibitors of human leukocyte elastase. 3. Stereospecific synthesis and structure—Activity relationships for 3,3-dialkylazetidin-2-ones. J. Med. Chem. 38, 2449-2462.

5. Gallin, J. I., Goldstein, I. M. & Snyderman, R. (1988) Inflammation. Chapter 41, Pathogenesis of rheumatoid arthritis: A disorder associated with dysfunctional rinnunoregulation. 751-774, ISBN 008167344-7.

6. Groutas, W. C. (1987) Inhibitors of leukocyte elastase and leukocyte cathepsin G. Agents for the treatment of emphysema and related ailments. Medicinal Researh Reviews, 7(2), 227-241.

7. Heck, H. L., Darby, W. L., Bhown, A., Miller, E. J., Bennet, J. C. (1985) Isolation, characterization, and amino terminal amino acid sequence of human leukocyte elastase from normal donors. Analytical Biochemistry, 149, 153-162.

8. Jackson, A. H., Hill, S. L., Afford, S. C., Stockley, R. A. (1984) Sputum soluble phase proteins and elastase activity in patients with cystic fibrosis. J. Respir. Dis. 65, 114-124.

9. Metcalf, J. A., Gallin, J. I., Nauseef, W. M., Root, R. K. (1986) Laboratory Manual of Neutrophil Function, Raven Press Ltd., New York.

10. Shephard, E. G., Anderson, R., Rosen, O., Myer, M. S., Fridkin, M., Strachan, A. F. & De Beer, F. C. (1990) Peptides generated from C-reactive protein by a neutrophil membrane protease. Amino acid sequence and effects of peptides on neutrophil oxidative metabolism and chemotaxis. J. Immunol., 145, 1469-1476.

11. Shephard, E. G., Kelly, S. L., Anderson, R. & Fridkin, M. (1992) Characterization of neutrophil-mediated degradation of human C-reactive protein and identification of the protease. Clin. Exp. Immunol., 87, 509-513.

12. Vachino, G., Heck, L. W., Gelfand, J. A., Kaplan, M. M., Burke, J. F., Berninger, R. W., McAdam, K. P. (1988) Inhibition of human neutrophil and Pseudomonas elastases by the amyloid P-component: A constituent of elastic fibers and amyloid deposits. J. Leukocyte Biol. 44, 529-534.

13. Yavin, E. J., Rosen, O., Pontet, M., Shephard, E. G., Fridkin, M. (1995) Proteolysis of hunan C-reactive protein by neutrophil-derived Iysosomal enzymes generates peptides which modulate neutrophil function: Implication to the anti-inflammatory mechanism. Letters in Peptide Science, 2, 7-16.

1. A peptide capable of inhibiting in vitro the enzymatic activity of human Leukocyte Elastase (hLE) and/or of human Cathepsin G (hCG), said peptide being selected from:
(i) a core peptide corresponding to positions 89-96 of the sequence of human C-reactive protein (CRP) of the formula:
Val₈₉-Thr-Val-Ala-Pro-Val-His-Ile₉₆
or a modification thereof characterized by:
(ii) substitution of Ile₉₆ by a hydrophobic amino acid residue;
(iii) substitution of His₉₅ by D-His or by a residue selected from Asp, Glu, Ser, Thr, Phe and Tyr, N-alkyl derivatives thereof and D-forms of the foregoing;
(iv) substitution of Val₉₄ by D-Val, or by a residue selected from Ala, His and Phe, and D-forms of the foregoing;
(v) substitution of Ala₉₂ by a hydrophobic amino acid residue;
(vi) substitution of Val₉₁ by Ala or Gly;
(vii) substitution of Thr9O by a residue selected from Asn, Asp, Gln, Glu, Ala, Val and Pro;
(viii) substitution of Val₈₉ by a hydrophobic amino acid residue;
(ix) a peptide obtained by elongation of a peptide (i) to (viii) at the N- and/or C-terminal;
(x) an amide of the C-terminal of a peptide (i) to (ix); and
(ix) an N-acyl derivative of a peptide (i) to (x).

2. A peptide according to claim 1 wherein the hydrophobic amino acid residue is selected from a residue comprising Leu, Ile, Val, Phe, Tyr, Nle and Nva.

3. A peptide according to claim 1(ix) wherein the peptide is elongated by additional amino acid residues at the N-terminal.

4. A peptide according to claim 3 wherein the additional amino acid residues constitute sequences of the human CRP.

5. An N-acyl peptide according to claim 1 (xi) wherein acyl is a radical R—X—CO—, wherein R is substituted or unsubstituted hydrocarbyl and X is a covalent bond, O, NH, or NHCO.

6. An N-acyl peptide according to claim 5 wherein R is optionally substituted alkanoyl or aroyl.

7. An N-acyl peptide according to claim 6 wherein the acyl radical is selected from octanoyl, monomethoxysuccinyl, carbobenzoxy (benzyl-O—CO—), acetylaminocaproyl, Fmoc (fluorenylmethoxycarbonyl), naphthyl-NH—CO— and adamantyl-NH—CO—.

8. A peptide according to any one of claims 1 to 7 selected from the sequences:
Val-Thr-Val-Ala-Pro-Val-His-Ile
Val-Thr-Val-Ala-Pro-Val-(D)His-Ile
Val-Thr-Val-Ala-Pro-(D)Val-His-Ile
Val-Thr-Val-Ala-Pro-(D)Val-(D)His-Ile
Val-Thr-Val-Ala-Pro-Val-Ser-Ile
Val-Thr-Val-Ala-Pro-Val-Phe-Ile
Val-Thr-Val-Ala-Pro-Val-His-Ile-NH₂
Val-Thr-Val-Ala-Pro-Val-His-Ile-Pro-NH₂
Val-Thr-Val-Ala-Pro-Phe-His-Ile-Pro-NH₂
Val-Thr-Val-Ala-Pro-Val-His-Ile-Pro-Pro-NH₂
MeOSuc-Val-Thr-Val-Ala-Pro-Val-His-Ile
MeOSuc-Phe-Val-Thr-Val-Ala-Pro-Val-His-Ile
Octanoyl-Val-Thr-Val-Ala-Pro-Val-His-Ile
Acetylaminocaproyl-Val-Thr-Val-Ala-Pro-Val-His-Ile
Adamantyl-NH-CO-Val-Thr-Val-Ala-Pro-Val-His-Ile
α-Naphthyl-NH-CO-Val-Thr-Val-Ala-Pro-Val-His-Ile
CBz-Val-Thr-Val-Ala-Pro-Val-His-Ile
CBz-Phe-Val-Thr-Val-Ala-Pro-ValHis-Ile
Fmoc-Val-Thr-Val-Ala-Pro-Val-His-Ile
wherein Cbz is carbobenzoxy, MeOSuc is monomethoxysuccinyl and Fmoc is 9-fluorenylmethoxycarbonyl.

9. A pharmaceutical composition comprising a CRP-derived peptide according to any one of claims 1 to 8 and a pharmaceutically acceptable carrier.

10. Use of a CRP-derived peptide according to any one of claims 1 to 8 for the preparation of a pharmaceutical composition for the treatment of chronic inflammatory conditions.

11. Use according to claim 10 wherein the chronic inflammatory condition is rheumatoid arthritis, pulmonary emphysema or cystic fibrosis.

12. A method for the treatment of a chronic inflammatory condition which comprises administering to a patient in need thereof an effective amount of a peptide according to any one of claims 1 to 8.

13. A method according to claim 12 wherein the chronic inflammatory condition is rheumatoid arthritis, pulmonary emphysema or cystic fibrosis.
